(19) United States
(12) Patent Application Publication
(10) Pub. No.: US 2014/0148730 A1
(43) Pub. Date: May 29, 2014

(54) PUNCTURING DEVICE FOR REMOVING ORGANIC SAMPLES

(71) Applicant: Hans-Peter Steiner, Graz (AT)

(72) Inventor: Hans-Peter Steiner, Graz (AT)

(21) Appl. No.: 14/131,199

(22) PCT Filed: Jun. 28, 2012
(86) PCT No.: PCT/EP2012/062536

§ 371 Date: Jan. 9, 2014

(30) Foreign Application Priority Data
Jul. 4, 2011 (AT) A 978/2011

(51) Int. Cl. A61B 10/02

(52) U.S. Cl. CPC A61B 10/0283
USPC 600/566

(57) ABSTRACT

A puncturing device for extracting organic samples, and which includes a puncturing cannula ending in a needle tip, the lumen of the puncturing cannula being designed as a suction channel, and a flushing line for supplying a flushing agent. Behind the needle tip of the puncturing cannula a single-lumen puncturing area is provided, which is followed by a double-lumen area departing from a connecting site at which the flushing line is fluid-tightly attached to the puncturing cannula. At a distal end of the flushing line is provided at least one transfer opening into the suction channel of the puncturing cannula.

### CROSS-REFERENCE TO RELATED APPLICATIONS

The present application is a National Stage Application of PCT International Application No. PCT/EP2012/062536 (filed on Jun. 28, 2012), under 35 U.S.C. §371, which claims priority to Austrian Patent Application No. A 978/2011 (filed on Jul. 4, 2011), which are each hereby incorporated by reference in their respective entireties.

### TECHNICAL FIELD

Embodiments relate to a puncturing device for extracting organic samples, such as egg cells for in-vitro fertilization, comprising a puncturing cannula ending in a needle tip, the lumen of the puncturing cannula being designed as a suction channel, and a flushing line for supplying a flushing agent, where behind the needle tip of the puncturing cannula a single-lumen puncturing area is provided, which is followed by a double-lumen area departing from a connecting site at which the flushing line is fluid-tightly attached to the puncturing cannula, and where at the distal end of the flushing line there is provided at least one transfer opening into the suction channel of the puncturing cannula.

### BACKGROUND

In the context of infecundity therapy using classical in-vitro fertilization (IVF), egg cells are obtained by puncturing the follicle immediately prior to expected ovulation. Maturing of the egg cells is assisted by means of hormone treatment, which will result in the simultaneous maturing of several egg cells and thus in a higher success rate of IVF. Follicle puncture is usually carried out using puncturing needles with an outer diameter of 1.4 mm to 1.5 mm and a length of 30 cm, with approximately 5 ml follicle fluid being aspirated from a follicle (diameter up to 2.5 cm).

In some cases, hormone treatment may lead to problems, e.g. for patients with PCO syndrome (polycystic ovaries) for whom it may cause the life-threatening complication of a so-called ovarian hyper-stimulation syndrome (OHSS). A new method, i.e. in-vitro maturation (IVM), avoids this complication.

In the IVM method follicle maturation is hormone-initiated in a spontaneous cycle without prior hormone treatment, usually between the 8^{th} and 10^{th} day of the cycle, when follicle size is between 8 mm and 10 mm, all follicles present after about 36 to 38 hours with sizes upwards of 3mm to 4 mm being punctured and aspirated under ultrasonic control. The egg cells are then matured for 24 hours in an incubator and, if a polar body is present, fertilized by means of the ICSI method.

Due to the small follicle size most of the conventional puncturing sets for extracting egg cells are not suitable for the IVM method.

It is known in the art to use puncturing devices with a flushing component, where by means of a three-way valve connected to a single-lumen puncture needle, flushing or aspiration may be alternatively effected depending on the position of the three-way valve. A disadvantage of such systems lies in the fact that they generally have a large dead volume corresponding to the volume between cannula tip and three-way valve of the puncturing device.

In order to minimize dead volume double-lumen puncturing cannulae have been used, one lumen being used for aspiration and the other for flushing of the follicle. The disadvantage of such double-lumen puncturing sets lies in a relatively large overall diameter, which makes such sets unsuitable for the IVM method.

From German Patent Publication No. DE 35 22 782 A1 there is known a double-lumen puncturing cannula for follicle puncture, which comprises an outer tube of essentially circular cross-section and an inner tube whose outer wall lies against the inner wall of the outer tube along a large section of its periphery. The inner tube has a small inwardly curved region of less than 90° of peripheral angle, which together with the outer tube forms a flushing channel. The remaining suction channel in the inner tube however impedes the free passage of the egg cells obtained by the puncturing due to the inwardly curved region. The curved region furthermore forms an edge in the entry opening at the needle tip, which will also impede the taking of organic samples.

Finally there is known from European Patent Publication No. EP 1 967 147 B1 a puncturing device for taking organic samples, in which a thinner single-lumen puncturing region is provided next to the needle tip, which is followed by a double-lumen region departing from a connecting site at which a flushing line is fluid-tightly attached to the puncturing cannula. In the double-lumen region the puncturing cannula lies inside the flushing line, thus forming a flushing channel between the outer wall of the puncturing cannula and the inner wall of the flushing line, which flushing channel is provided at its distal end with a transfer opening into the suction channel of the puncturing cannula.

While the single-lumen puncturing section of the cannula may be produced with a smaller outer diameter than is known for double-lumen needles, thus making follicle puncture less painful due to reduced needle thickness, a smaller needle diameter has the disadvantage that the manufacturing expense of giving the transfer opening from the flushing channel a sufficiently large diameter for optimum flushing is high. A transfer opening with an inner diameter that is large relative to the needle diameter may furthermore weaken the needle structure in this area, which may cause breakage or bending of the puncturing cannula.

### SUMMARY

It is an object of embodiments to further develop a puncturing device for taking an organic sample, e.g. an egg cell, in such a way that it will also be well suited for in-vitro maturation (IVM), in addition to conventional IVF, and to create a puncturing set that can be realized in a simple and economical way.

In accordance with embodiments, this object is achieved by proposing that the puncturing cannula have a smaller diameter in the puncturing area than in the proximally adjacent double-lumen area, the at least one transfer opening being provided at the distal end of the double-lumen area. The area containing the transfer opening thus has greater diameter than the single-lumen area at the needle tip, thus permitting the use of needles with very small outer diameters for puncturing.

Preferably, the puncturing cannula comprises a thin puncturing needle (for instance a 22-gauge needle) and a proximally adjacent connecting cannula (for instance an 18-gauge cannula), the proximal end of the puncturing needle extending into the connecting cannula. The outer diameter of the puncturing needle (e.g. 0.72 mm) matches the inner diameter of the connecting cannula (0.84 mm).

In accordance with embodiments, the proximal end of the puncturing cannula may be attached to the connecting cannula by clamping, bonding or welding (e.g. laser welding).

In the case of bonded attachment the puncturing needle may be made of steel and the connecting cannula of plastics material, which would significantly reduce production cost.

It is furthermore possible to shrink the connecting cannula onto the end of the puncturing needle. In that case the puncturing needle has an outer diameter slightly larger than the inner diameter of the connecting cannula. Following heating of the connecting cannula and/or cooling of the puncturing needle (for example by liquid nitrogen) the two parts are stuck together and are rigidly attached to each other after temperature equalization.

The flushing line of the puncturing device may be a flexible flushing tube having a diameter reduction or a shrink-on connection or a bonded connection at the connecting site to the puncturing cannula.

### DRAWINGS

The invention will now be described in more detail with reference to the enclosed drawings, in which:

FIG. 1 illustrates a puncturing device in accordance with embodiments in a longitudinal section.

FIG. 2 illustrates a detail II from FIG. 1 in an enlarged view.

### DESCRIPTION

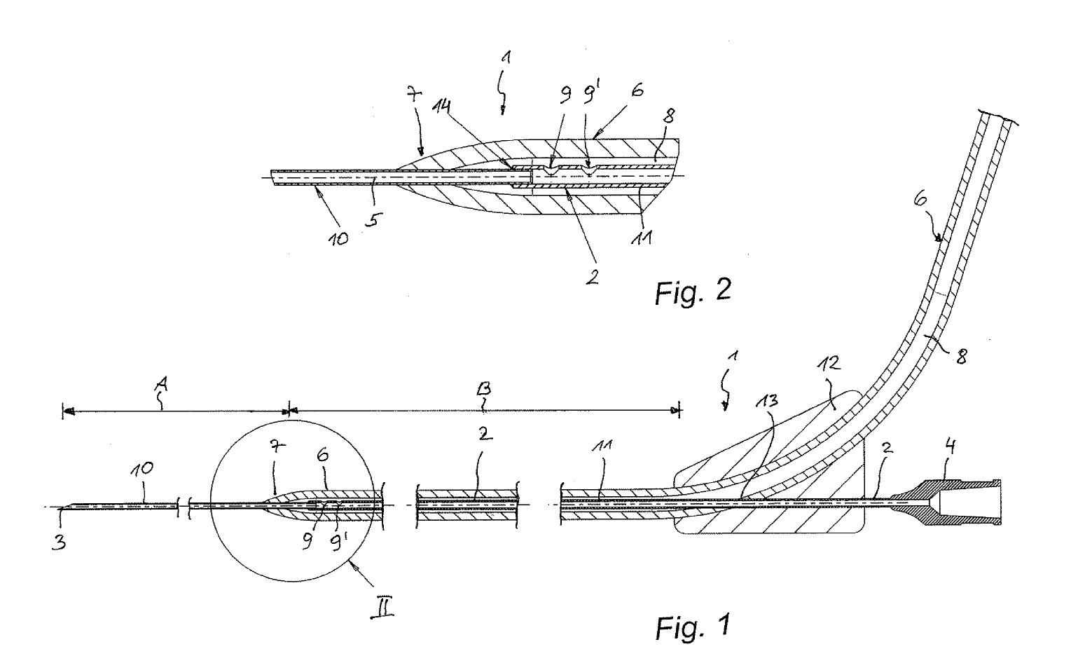

The puncturing device 1 as in FIG. 1 with detail in FIG. 2 is used for taking an organic sample and has a puncturing cannula 2 with a sharpened needle tip 3 at its distal end and a Luer connector 4 at its proximal end, where via a connecting tube a container may be attached for the organic sample, for instance an egg cell. As is known from the initially cited EP 1 967 147 B1, the container is connected to a vacuum source, which generates the partial vacuum needed for sample extraction in the lumen of the puncturing cannula 2 configured as a suction channel 5.

There is furthermore provided a flushing line 6 designed as a flexible tube, which supplies a flushing agent, for instance for follicle flushing. A plunger syringe acting as a container for the flushing agent may be connected to the flushing line 6, and an optimum result of follicle flushing may be obtained by simultaneous coordinated suction and flushing activity (see, EP 1 967 147 B1).

The flushing line 6 comprises the puncturing cannula 2 and is fluid-tightly attached to it at a connecting site 7 at a distance of 6-9 cm from the needle tip 3. Between the outer wall of the puncturing cannula 2 and the inner wall of the flushing line 6 there is formed an annular region of the flushing channel 8, which has two transfer openings 9, 9′ into the suction channel 5 at the distal end (see FIG. 2). In the puncturing area A the puncturing cannula 2 has a smaller diameter than in the proximally adjacent double-lumen area B, the transfer opening 9, 9′ being located at the distal end of the double-lumen area B.

The design in accordance with embodiments provides a thin single-lumen puncturing area A adjacent to the needle tip 3, which is particularly suitable for egg cell extraction, while the transfer openings 9, 9′ are located in an area of larger diameter which makes larger cross-sections possible.

As illustrated in FIGS. 1 and 2, the puncturing cannula 2 essentially consists of a relatively thin puncturing needle 10 with a needle tip 3 and a proximally adjacent connecting cannula 11, with the proximal end of the puncturing needle 10 extending into the connecting cannula 11. The outer diameter of the puncturing needle 10 may thus be optimally adapted to the inner diameter of the connecting cannula 11 and may be attached by clamping, shrinking, bonding or welding.

The puncturing needle 10 may for instance be made of metal, preferably a steel alloy, and the connecting cannula 11 may be made of plastics material. It is also possible to use metal, preferably a steel alloy, for both the puncturing needle 10 and the connecting cannula 11 and to join these parts by welding, for instance laser welding 14.

The connecting cannula 11 may be provided with two or more transfer openings 9, 9′ in the distal area, which preferably are located one behind the other in axial direction. This will increase the flow cross-section of the transfer openings without excessive weakening of the needle structure in this area.

The flushing line 6 preferably is realized as a flexible flushing tube, which has a reduced diameter or is attached by shrinking or bonding at the site 7 where it connects to the puncturing cannula 2. The puncturing cannula 2 may be provided with a grip element 12 and may enter the flushing line 6 in this area via a puncture 13. The flexible flushing tube will seal the puncturing cannula 2 so as to be fluid-tight at the puncture 13.

1-10. (canceled)

11. A puncturing device for taking organic samples, comprising:
a puncturing cannula tapering to a needle tip, the puncturing cannula having a lumen configured as a suction channel;
a flushing line configured to supply a flushing agent, wherein adjacent to the needle tip is provided a single-lumen puncturing area followed by a double-lumen area departing from a connecting site where the flushing line is attached in a fluidically sealed manner to the puncturing cannula;
at least one transfer opening located at the distal end of the double-lumen area and opening into the suction channel provided at the distal end of the flushing line, wherein the puncturing cannula has a first diameter in the puncturing area that is less than a second diameter of the puncturing cannula in the double-lumen area.

12. The puncturing device of claim 11, wherein the puncturing cannula further comprises a connecting cannula into which extends a proximal end of a puncturing needle.

13. The puncturing device of claim 12, wherein the outer diameter of the puncturing needle is equal to the inner diameter of the connecting cannula.

14. The puncturing device of claim 12, wherein the proximal end of the puncturing needle is attached to the connecting cannula by at least one of clamping, shrinking and bonding.

15. The puncturing device of claim 14, wherein the puncturing needle is made of metal and the connecting cannula is made of a plastic material.

16. The puncturing device of claim 14, wherein the puncturing needle is made of a steel alloy and the connecting cannula is made of a plastic material.

17. The puncturing device of claim 12, wherein the puncturing needle and the connecting cannula are both made of metal.

18. The puncturing device of claim 17, wherein the puncturing needle and the connecting cannula are joined by welding.

19. The puncturing device of claim 12, wherein the puncturing needle and the connecting cannula are both made of a steel alloy.

20. The puncturing device of claim 19, wherein the puncturing needle and the connecting cannula are joined by welding.

21. The puncturing device of claim 12, wherein the connecting cannula is provided at a distal area with two or more transfer openings into the suction channel.

22. The puncturing device of claim 21, wherein the transfer openings are disposed one behind the other in an axial direction.

23. The puncturing device of claim 11, wherein the flushing line is configured as a flexible flushing tube, which at a connecting site to the puncturing cannula has a reduced diameter, a shrink-on connection or a bonded connection.

24. The puncturing device of claim 23, wherein the puncturing cannula is provided with a grip element and enters into the flushing line in an area of the grip element via a puncture, which is fluidically sealed by the flexible flushing tube.

25. A puncturing device for extracting eggs for in-vitro fertilization, the puncturing device comprising:
a puncturing cannula including a needle tip and a lumen having a suction channel;
a flushing line to supply a flushing agent, wherein a single-lumen puncturing area is provided adjacent to the needle tip, followed by a double-lumen area departing from a connecting site where the flushing line is attached in a fluidically sealed manner to the puncturing cannula;
a transfer opening located at the distal end of the double-lumen area and opening into the suction channel provided at the distal end of the flushing line,
wherein the puncturing cannula has a first diameter in the puncturing area that is less than a second diameter of the puncturing cannula in the double-lumen area.
